(19) United States
(12) Patent Application Publication
(10) Pub. No.: US 2025/0014221 A1
(43) Pub. Date: Jan. 9, 2025

(54) PATIENT-ACCESSIBLE CALIBRATION OF VIDEO EYETRACKING APPARATUS

(71) Applicant: New York University, New York, NY (US)

(72) Inventor: Todd Hudson, New York, NY (US)

(73) Assignee: New York University, New York, NY (US)

(21) Appl. No.: 18/765,178

(22) Filed: Jul. 5, 2024

Related U.S. Application Data
(60) Provisional application No. 63/525,604, filed on Jul. 7, 2023.

(51) Int. Cl. G06T 7/80; A61B 3/00; A61B 3/02; A61B 3/113; A61B 5/16; G06T 7/246; G06T 7/55; G06T 7/73

(52) U.S. Cl. CPC G06T 7/80; A61B 3/0091; A61B 3/02; A61B 3/113; A61B 5/163; G06T 7/246; G06T 7/55; G06T 7/73; G06T 2207/30041

(57) ABSTRACT

A system includes one or more processors and one or more memories having instructions stored thereon which, when executed by the one or more processors, cause the one or more processors to perform operations include receiving detected ocular landmarks in a subject, determining ocular parameters based on the detected ocular landmarks, and determining, based on the ocular parameters, a coarse calibration output. The operations include receiving, eyetracking data relating to an eye movement made by the subject, determining a landing position of the eye at the end of the eye movement, sending a signal to display a target at the landing position on a screen, and receiving, from the subject, an input including a response relating the landing position to a gaze parameter of the subject. The gaze parameter is associated with the eye movement. The operations include updating the coarse calibration output based on the input.

### CROSS-REFERENCE TO RELATED PATENT APPLICATION

This application claims the benefit of priority to U.S. Provisional Patent App. No. 63/525,604, filed on Jul. 7, 2023, the entirety of which is incorporated by reference herein.

### TECHNICAL FIELD

The present disclosure relates generally to the calibration of eyetracking cameras, to asynchronous camera array and methods for reduced noise and anti-aliased 3D video acquisition in eyetracking applications.

### BACKGROUND

Many neurodegenerative diseases (e.g., Alzheimer's Disease, Parkinson Disease, Multiple Sclerosis (“MS”), Progressive Supranuclear Palsy (“PSP”), etc.) cause altered ocular motor function (e.g., abnormal eye movements). These eye movements can serve as a basis for diagnosis, grading of disease severity, as well as a sensitive marker of the response to therapeutics. Eyetracking, in both laboratory and clinic environments, can provide valuable insight into neurological disorders, and allow quantitative description of the patterns and presence of pathological ocular motor control in many neurological disorders. Thus, it is important to characterize eye movements with accuracy and precision, High-precision, objective eyetracking can provide the spatial and temporal resolution required to characterize subtle changes in ocular motor function. For example, high-precision eye movement recording can help improve early detection, particularly in the presymptomatic and prodromal stages of neurodegenerative disorders,

Eyetracking research has emerged using video-oculographic (“VOG”) techniques, with increasing interest in capturing gaze metrics in a variety of applications. Many eyetracking applications are able to employ “calibration-free” eyetracking, in which a simplified model of the eye (spherical eye, rotational symmetric lens and cornea, etc.) is used along with anthropomorphic averages of ocular parameters to predict gaze location. Although calibration-free eyetracking is sufficient for inferring the general direction of gaze, these systems suffer from inter-subject variable errors of between 3° and 10°. And while part of this error is theoretically correctable with a more accurate eye model, a significant portion is uncorrectable because it is due to individual differences in eye shape, refractive power at the cornea/lens, and retinal structure. This last in particular gives rise to on average a 3°-8° horizontal difference and another 1°-3° vertical difference between the optical (or pupillary) and visual/gaze axes (FIGS. 2A-2B) that skews the results of any data derived from calibration-free systems by an unknown amount. Consequently, only calibrated eyetracking is considered sufficiently precise for laboratory and clinical measurements. Calibration creates a mapping between eye and gaze direction.

However, current procedures for calibrating eyetracking equipment can be challenging for clinical populations, such as neurologically impaired populations. The calibration procedure for most widely available and convenient eyetracking technology (video-oculography) is often too difficult for patients to perform successfully. As one example, while many studies have been done of aspects of diseases such as examining the ocular motor sequelae of Parkinson disease and PSP and other supranuclear gaze palsies, as well as in stroke, concussion, and multiple sclerosis, these studies are limited in their usefulness due to difficulties with calibration and the resulting reduced recording data quality in these groups. As a result, neurologically impaired patients are routinely excluded from ocular-motor research due to challenges experienced while calibrating eyetracking equipment that either make it difficult to perform calibration successfully, or yield poor calibration of limited usefulness. These challenges are particularly prevalent in elderly populations, and in patients with fixational instability (nystagmus, square wave intrusions), slow eye movements, reduced oculomotor range, and attentional difficulties.

Excluding neurologically impaired patients from eyetracking research has three primary negative effects: (1) limited progress toward understanding disease states; (2) impeded detection, diagnosis, and tracking of disease progression; and (3) inadequate stratification of patient sub-groups in clinical trials. The aforementioned challenges may also contribute to poor calibration of eyetracking equipment, and in some instances, complete failure to calibrate eyetracking equipment. Despite the development of several novel mathematical and methodological tools to acquire and process eye movement data, these difficulties persist, as data quality is limited by calibration quality. Thus, to obtain quality eyetracking data in clinical populations, such as neurologically impaired populations, an improved calibration procedure is needed.

Three main obstacles to calibration exist: (1) impaired ability to fixate for prolonged periods (i.e., to inhibit normal visual exploration); (2) impaired cognitive attention; and (3) pathological eye movements. First, calibration of modern video-oculographic equipment requires that patients perform a series of relatively long, stable fixations of targets positioned on a screen. However, stable fixation for prolonged periods is challenging. Humans naturally make 2-4 saccades (e.g., a rapid eye movement between fixation points) per second, and inhibiting these exploratory movements can be difficult or impossible for patients, such as the elderly, neurologically compromised, and in some instances, healthy controls. Difficulty may be due to cognitive deficits, pathological eye movements that intrude on fixation (nystagmus, saccadic intrusions, etc.), or difficulty suppressing exploratory eye movements, which normally occur at 2-4 Hz.

Second, the cognitive attention required to fixate over long periods can pose a problem for some patients, even those capable of suppressing visual exploration. Third, importantly, unintended eye movements (saccadic intrusions, nystagmus, etc.), slowed, delayed, or restricted in range occur in many neurological disorders. A calibration procedure involving freely viewing a screen, regardless of unintended eye behavior, and requiring only brief bursts of attention, is needed to address these issues.

### SUMMARY

One embodiment relates to a system including one or more processors and one or more memories operatively coupled to the one or more processors. The one or more memories have instructions stored thereon which, when executed by the one or more processors, cause the one or more processors to perform operations. The operations cause the one or more processors to detect ocular landmarks of a subject's eye, determine ocular image parameters based on the ocular landmarks, determine a coarse calibration output associated with the ocular image parameters, detect a first eye movement made by the subject, the first eye movement associated with a first gaze direction, and determine a first landing position of the eye at the end of the first eye movement, the ocular landmarks at the first landing position associated a first gaze location on a screen. The instructions include code to cause the one or more processors to display, on the screen, a target at the first gaze location, receive, in response to the target being displayed at the first gaze location, a first input from the subject, the first input including a response relating the first landing position to the first gaze direction, and update the coarse calibration output based on the first input.

One embodiment relates to a system including one or more processors and one or more memories operatively coupled to the one or more processors. The one or more memories have instructions stored thereon which, when executed by the one or more processors, cause the one or more processors to perform operations. The operations include receiving images, the images comprising two or more image sets, each image set acquired from a different camera. The operations include determining estimates of camera projections in real space and localizing current features, determining a state of image features by comparing characteristics of present images to previously acquired images, advancing the state of image features for the previously acquired images based on state parameters corresponding to the previously acquired images and determining an estimated current state of the image features for the previously acquired images, determining estimated current positions of the image features estimated current state of the image features for the previously acquired images; determining image disparities for each image feature by comparing current positions of the image features in an image set of the two or more image sets to estimated current positions of the same image features in a different image set of the two or more image sets, identifying corresponding image features within different images of different image sets, determining spatial offsets of current positions of the image features and estimated current positions of the image features of the corresponding image features in each image set, generating a disparity map from the image disparities, and generating a depth map from the disparity map.

One embodiment relates to a system including one or more processors and one or more memories operatively coupled to the one or more processors. The one or more memories have instructions stored thereon which, when executed by the one or more processors, cause the one or more processors to perform operations. The operations include receiving, from two or more cameras, eyetracking parameters associated with pupil, limbus, and corneal reflection of an eye. The operations include determining a gaze estimate based on the eyetracking parameters associated with pupil, limbus, and corneal reflection, determining a depth map of a corneal surface of the eye, determining a gaze estimate based on the depth map, and combining the gaze estimate based on the eyetracking parameters associated with pupil, limbus, and corneal reflection and the gaze estimate based on the depth map, forming a fused gaze estimate.

### BRIEF DESCRIPTION OF THE FIGURES

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

FIGS. 1A-1C show a diagram comparing three types of calibration and gaze computation methods: traditional (FIG. 1A), hybrid mapping (FIG. 1B), and hybrid model (FIG. 1C).

FIGS. 2A-2B show a two-stage calibration process, according to one embodiment. FIG. 2A shows Stage I, where an approximation to the gaze axis of the eye is computed geometrically from camera images using the eye's optic axis as a proxy for the gaze axis (ω is the orientation of the optical axis of the eye; diagram not to scale). FIG. 2B shows Stage II, where the calibration is refined, improving the gaze estimate, by measuring gaze estimation errors at a range of on-screen fixations.

FIG. 3A shows eye data overlaid on 6×6 grid positions (black circles), where grey dots indicate gaze locations while the grid was flashed. FIG. 3B shows named character position as a function of eye position (circles); dashed line represents ideal result.

FIG. 4 shows the predicted calibration precision (1/stdev) for traditional and new calibration. Saccades were simulated at 3-4 Hz and computed calibration precision from an amount of data equivalent to 1.1 s per traditional calibration target (and assuming that only ¼ of all fixations are captured by the new algorithm).

FIG. 5A shows spatial differences between gaze and grid characters. FIG. 5B shows histograms of sapling distributions of errors between gaze and named letter. Spatial errors are uniform without letter naming errors. A letter naming error (named letter not closest to gaze) in half of all fixations yields a triangular sampling distribution (first convolution of the errorless distribution). FIG. 5C shows sampling distribution of the mean of 50 data, assuming 50% errors in letter naming.

FIG. 6 shows a generative model of binary up/down or left/right responses in a direction-report method, according to one embodiment.

FIGS. 7A-7B show inverse calibration in the dot method (FIG. 7A) and in the grid method (FIG. 7B).

FIG. 8 is a diagram showing how an algorithm processes images taken from various cameras at specific time intervals.

FIGS. 9A-9C are diagrams illustrating the sensor fusion approach to estimating gaze for pupil (FIG. 9A), limbus (FIG. 9B), and the disparity (FIG. 9C).

FIG. 10 is a diagram showing a method of determining a coarse calibration and updating the coarse calibration, according to an embodiment.

FIG. 11 is a diagram showing a method of determining a gaze estimate, according to an embodiment.

FIG. 12 is a diagram showing a method of determining a depth map from two or more cameras that acquire images asynchronously, according to one embodiment.

FIG. 13 is a diagram showing a method of combining a depth map of a corneal surface with estimates that rely on pupil position, corneal reflection, and limbus tracking, according to one embodiment.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate to systems and methods relating to eyetracking. Calibration is required to ensure accurate eyetracking results. The embodiments described herein include a calibration technique for high-precision objective eyetracking systems, such as those used in clinical and experimental neurology. The calibration technique accounts for a range of patients, with varying abilities and characteristics, and provides an accessible and precise calibration procedure. Embodiments described herein prioritize patient comfort by reducing stress and/or frustration associated with the calibration procedure. The calibration procedure is self-paced and adaptive to patient needs, allowing patients to more easily produce correct fixations. Embodiments described herein further bypass the need for prolonged, stable fixation typically required in calibration procedures. Therefore, a calibration technique can be employed that is more reliably successful in a broad range of patient populations, and allows for the acquisition of high-fidelity, clinically useful eye movement recordings in patients.

Standard, or typical, calibration procedures include automatic calibration and manual calibration. Automatic calibration relies on a model of the eye to compute gaze direction (e.g., the normal to the pupil plane is taken to be the gaze direction). The model of the eye used in automatic calibration differs between algorithms. However, generally, the models rely on assumptions that are often not correct in the general population, or inaccurate in patients. Models generally assume that the eye is a sphere, and that the rotation axes of the eye pass through the center of that sphere. It is further typically assumed that the eyes are either oriented parallel to one another, or both directed to the same point in. An important assumption made in automatic calibration procedures is that the gaze axis of the eye is coincident with the optical axis. These assumptions are usually false, but to varying degrees depending on each individual's eye, and are particularly likely to be an inaccurate assumption in neurological patients, those with retinal dystrophies, with MS, or other retinal damage.

Manual calibration requires the user to fixate pre-defined locations in physical space. By obtaining uncalibrated data while fixating known 3D locations in space, it is possible to compute a mapping between images and gaze locations in space, and interpolate between these. An assumption that is always made during manual calibration procedures is that the user is truly fixating the pre-defined spatial locations (usually points on a computer screen) they are asked to fixate. This is often an incorrect assumption, as the user may briefly look to another point in space and this eye position is mistaken for fixation of the target. Another assumption often made during manual calibration is that both eyes are directed to the same point in space, which is never true for individuals with strabismus. This assumption can cause potentially large and variable errors in the final results of eyetracking.

The embodiments described herein provide an alternative to standard (e.g., typical) calibration procedures, and is designed to account for patients, such as those with neurologically compromised patients, who cannot maintain prolonged fixation, have reduced ocular range of motion, and/or have involuntary eye movements. The embodiments described herein further provide enhanced accuracy, precision, and reliability relative to existing calibration procedures. In particular, embodiments described herein relate to free-viewing (i.e., without mandating fixation of specific screen locations) calibration. Free-viewing calibration does not rely on prolonged fixations at pre-specified screen locations. Rather, it combines “calibration-free” methods that rely on a simplified model of the eye to approximate its optical axis, and an inversion of the usual “manual calibration” technique used for high-precision eyetracking. During free-viewing, raw data from naturally occurring fixations are recorded. Concurrently, a target is briefly flashed onscreen, and the subject reports the location of the flashed target relative to gaze. Multiple repetitions of this basic procedure allow calibration mappings to be precisely and efficiently computed. In one embodiment, the method takes a model-based estimate of the eye's optical axis as its prior estimate of the gaze axis, with subsequent updating (e.g., Bayesian updating, non-Bayesian updating, etc.) based on gaze data used to correct that prior estimate. Some embodiments of this method take the flashed targets near free-viewing gaze fixations to elicit one of several possible post-flash responses that underly Bayesian updates.

Free-viewing calibration is a two-tiered technique that inverts standard calibration procedures. The first tier creates a low-fidelity calibration based on a simplified eye model using anthropomorphic averages. The second tier uses targets flashed briefly at the current gaze estimate, allowing participants to essentially correct the underlying calibration with post-flash responses. A subject responds to a flashed stimulus to iteratively improve the calibration. This calibration technique corrects important shortcomings in standard calibration. In standard calibration it is simply assumed that fixations are accurately centered on calibration targets. This approach presents problems as, while fixation errors distort the calibration, no independent verification of fixation accuracy is available from calibration data. Distortion is also difficult to compensate using the pattern of other calibration targets because only ˜10 targets are fixated. Additionally, if any targets are outside a patient's ocular range of motion, traditional calibration fails, something that can present severe limitations for individuals with certain eye conditions, including those associated with aging. Inverting the order and asking subjects to respond to targets presented at estimated gaze-center during free-viewing fixations, automatically verifies gaze estimates. Free-viewing calibration also naturally matches ocular motility because targets are placed near natural gaze (rather than forcing subjects to view pre-set screen positions), and therefore automatically protects against failed calibrations due to reduced motility.

In some embodiments, the systems and methods described herein includes a calibration procedure which relies on targets flashed during free viewing of the screen, and a post-flash response. This bypasses the requirement (which is also the difficulty that typically excludes patient populations described above) that subjects fixate pre-selected target locations. The systems and methods described herein may utilize various alternatives for post-flash responses. For example, described herein are three alternatives, all relative to conventional manual or automatic calibrations: (1) saccadic-report method includes a “+” target flashed onscreen at the current system estimate of fixation, and observers being instructed to make a saccade to the target, indicating the estimation error; (2) direction-report method includes subjects reporting (via joystick, saccade, verbally, etc.) the vertical and horizontal error of flashed “+” targets relative to fixation when it was flashed; and (3) letter-report method a character grid flashed at system-estimated fixation, and subjects verbally reporting the letter closest to gaze center.

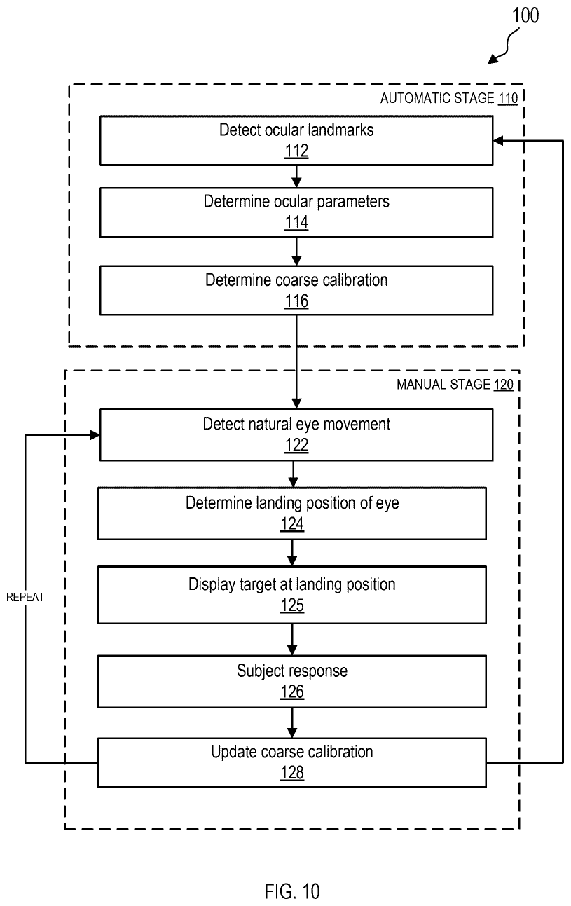

Referring to FIG. 10, a method 100 of calibration is shown. 110 is an automatic calibration stage. In some embodiments, an initial coarse calibration is computed from a model of the eye, allowing target placement on a screen during fixation at a gaze estimate. In some embodiments, a coarse calibration mapping is obtained. In some embodiments, an approximation to the gaze axis of the eye is computed geometrically from camera images, using the eye's optic axis as a proxy for the gaze axis. In some embodiments, an adaptive threshold for saccade detection is determined. In some embodiments, the coarse calibration is determined using geometry relating to corneal reflections, entrance pupil position, and the optic axis. In some embodiments, gaze is initially predicted based on the geometry of the eye and entrance pupil relative to the eye's optic axis.

At 112, ocular landmarks in camera images (e.g., pupil, corneal surface, glint, iris, etc.) are detected. In some embodiments, ocular landmarks are detected by a camera. At 114, ocular image parameters are determined based on the ocular landmarks. At 116, a coarse calibration output associated with the ocular parameters is determined. In some embodiments, the coarse calibration output including mapping between camera images and associated with the ocular image parameters.

120 is an automatic calibration stage. At 120, the coarse calibration from 110 is refined, improving the gaze estimate by measuring gaze estimation errors at a range of onscreen fixations. Subjects will engage in “free-viewing,” by looking at a screen, which may contain a static or dynamic image. In comparison to typical calibration procedures, the subject is allowed to make natural eye movements, scanning the screen. Raw data from naturally occurring fixations are recorded. A target (e.g., a circular target, crosshairs, letter-grid, etc.) is briefly flashed onscreen at the gaze estimate, and the subject provides an error signal (e.g., post-target response, location of the flashed target relative to gaze, etc.) that the algorithm uses to correct the calibration, as well as future gaze estimates. Multiple repetitions of 120 allow calibration mappings to be more precisely determined. In some embodiments, the coarse calibration mapping obtained in 110 is utilized as the Bayesian prior. In some embodiments, at 120, the model of the eye used in 110 is corrected using data from an error-report method (e.g., saccadic-report method, direction-report method, letter-report method, etc.). Bayesian updating is used to estimate the separation of gaze vs. optic axes of the eye at the anterior nodal point, marginalizing over all other model parameters. In some embodiments, a discrete set of screen calibration points are used to construct a mapping between the pupil-to-glint vector and gaze direction.

At 122, natural eye movement, such as a saccade, in a subject is detected. The first eye movement is associated with a first gaze direction. At 124, a landing position of the eye at the end of the first eye movement is determined. Ocular landmarks at the first landing position are associated with a first gaze location on a screen. At 125, a target at the first gaze location is displayed on the screen. At 126, in response to the target being displayed at the first gaze location, the subject provides a manual response indicating the target location relative to a gaze parameter, such as the first gaze direction. At 128, the coarse calibration generated at 116 is updated based on the manual response. Gaze may be modeled using the geometry relating pupil position and glints (from LEDs illuminating the eye) to the eye's optic axis. One method of updating the coarse calibration data is to use calibration data to refine the model of the eye (“model update”; e.g., incrementally correcting the eye model parameters, including that representing the angle separating optic and gaze axes). Alternatively, the model of the eye can provide initial predictions for a mapping of eye image features (such as pupil-glint positions) to onscreen gaze (“mapping update”), and this mapping (now divorced from the eye model) is updated following each fixation/target-flash/report cycle.

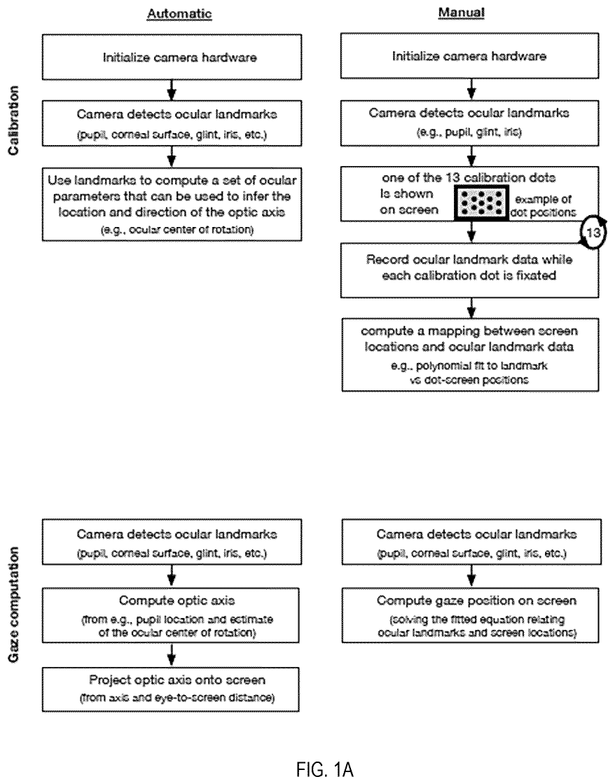

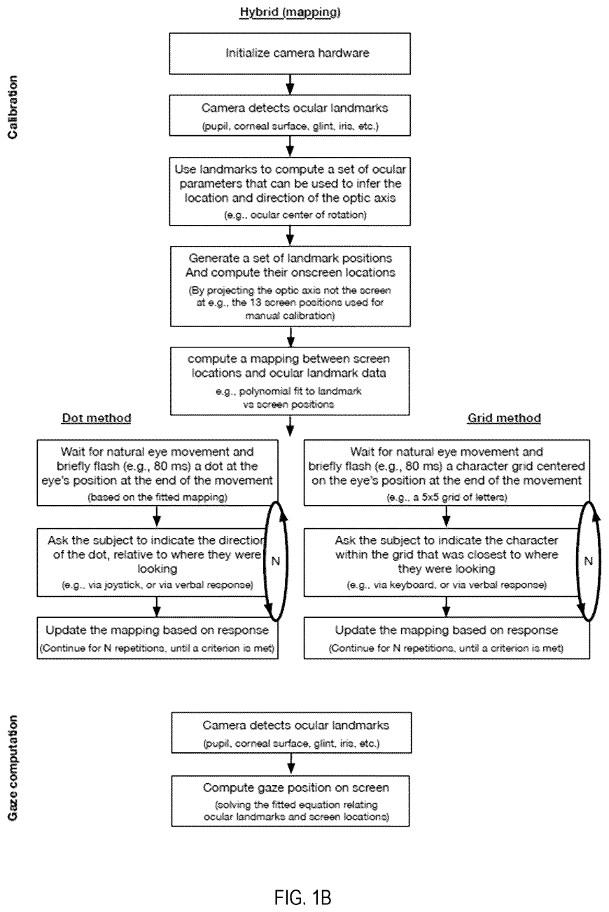

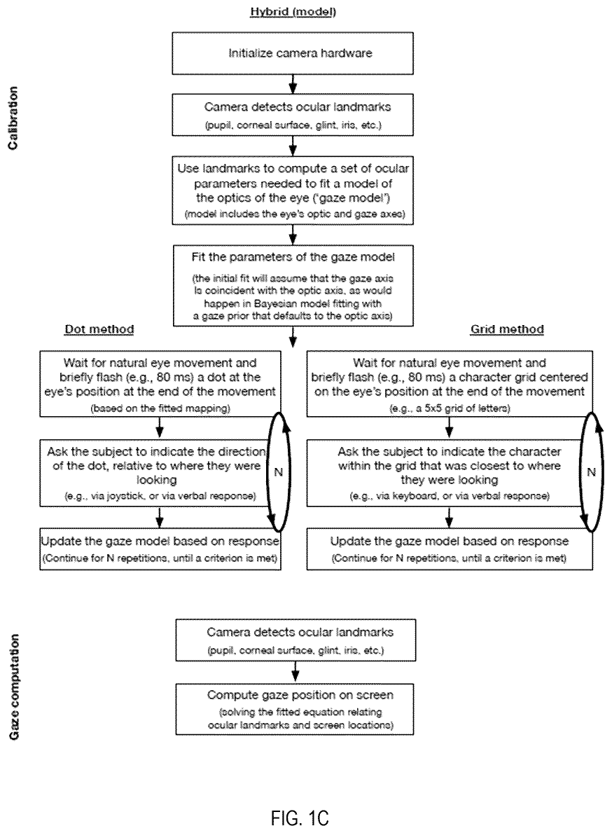

In one method 100, the process progresses from “automatic” to “manual” in succession, using the former as a coarse approximation (i.e., Bayesian prior) that is further refined (Bayesian and/or non-Bayesian updating) by data acquired during the second (manual) stage (see FIGS. 1A-1C). This coarse-to-fine progression provides information allowing: (a) measurement of ocular parameters needed to compute gaze direction (the elements of a parametric “gaze model”); (b) construction of a mapping between camera image and onscreen gaze position (best-fit parametric function through the image parameter vs. screen position multidimensional space; piecewise empirical scaling to map the multidimensional image parameter space onto screen position); and/or (c) a weighted combination of both.

The manual calibration phase 120 “inverts” the typical manual calibration procedure. In a typical manual calibration, there are pre-set screen locations that must be fixated. If a patient has impaired ocular motility and cannot fixate one or more of these locations, the “typical” procedure cannot be completed (or, must be attempted multiple times while modifying the screen locations in an attempt to accommodate the deficit). The inverted manual calibration procedure does not require fixation of any particular screen locations. Instead, the subject is allowed to make natural ‘free-viewing’ eye movements, scanning the screen. Using the coarse calibration afforded by the automatic calibration stage 110, one can track the progression (particularly, the initial portion) of each saccade and implement the following “inverse” algorithm (see FIGS. 7A-B):
a. Wait for a saccade (for example, identified by speed of eye position change or variance of position estimate);
b. Predict the landing position of the eye based on the saccade position and/or velocity profile during the initial portion of the saccade;
c. Generate a target (or target grid of characters) centered at the predicted landing position;
d. Present the target (or grid of characters) for 10-110 ms (or variably till the next saccade is initiated) following saccade termination;
e. After the target is turned off, user responds with either:
   i. the direction of the target relative to gaze direction (e.g., joystick, saccade); or
   ii. the grid character closest to the center of gaze (e.g., verbal response); and
f. Manual responses are used to update the coarse calibration obtained in the automatic calibration stage and used in (a) and (b) of this algorithm.
g. The process (a)-(f) are repeated until a desired calibration precision is achieved.

The method 100 can quickly correct the errors in automatic calibration, and in particular, will implicitly or explicitly determine the offset between optical and gaze axes of the eye and allow gaze to be tracked more accurately than can be accomplished with manual calibration alone, but be computed more quickly, easily, and reliably than conventual manual calibration. The method 100 also allows for multiple types of calibration to be computed and updated simultaneously, allowing the most precise, or in some embodiments, a fusion of calibrations, to be used for tracking.

It should be appreciated that the particular type of graphical display provided on the screen may be varied, for example, displaying flashes of light, drifting gratings, drifting sparkles or dots to prompt natural eye movements to a variety of screen positions.

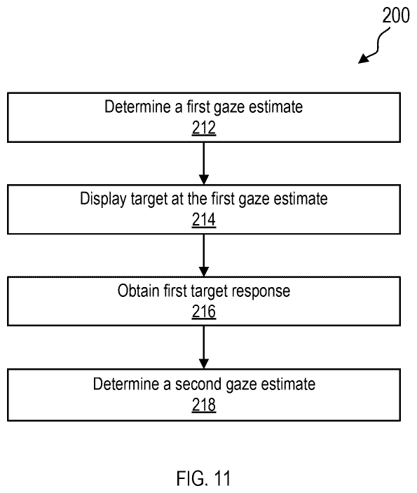

Referring to FIG. 11, a method 200 is shown. At 212, a first gaze estimate is determined based on an optic axis of a subject. At 214, a target is displayed on a screen facing the subject, the target is positioned at the first gaze estimate. The subject provides a first target response measuring gaze estimation error associated with the first gaze estimate. At 218 a second gaze estimate is determined by updating the first gaze estimate based on the first target response.

Predictive 3D from Non-Simultaneous and/or Non-Uniform Image Acquisition.

Embodiments described herein also relate to systems and methods relating to techniques of determining 3D depth maps from two or more cameras that acquire images asynchronously. In some embodiments, the two or more cameras further acquire images non-uniformly. This technique takes images acquired at previous times and forward-projects (e.g., predicts) the image features into current time The time-offset of camera images is accounted with predictive computations estimating (e.g., Bayesian predictive estimation) camera image feature locations from previously captured images, forward-predicted to the current time, which are used for disparity computations in conjunction with the camera image captured at the current time. Once all camera images, including images acquired at previous times and a current image, are forward-projected to the present time, they may be combined via standard techniques for stereo vision. In some embodiments, single previous camera images are forward-predicted and combined with a current camera image to compute disparity maps. In some embodiments, multiple previous camera images are forward-predicted to current time and combined both with the current camera image and with one another. The disparity maps computed from multiple combinations of cameras are fused via sensor fusion techniques (e.g., inverse-variance weighted). The resulting depth-maps can be fused for a final overall estimate of depth across camera views.

Predictive 3D from non-simultaneous image acquisition may be used in a range of applications, including eyetracking. Stereo depth is typically computed from simultaneous images obtained from two cameras displaced horizontally. This is the case in biological vision with human eyes displaced laterally (horizontally) with an inter-pupillary distance of about 6 cm. In some embodiments, it is assumed to be temporally non-uniform (not necessarily with equal temporal offset intervals) image acquisition amongst N spatially displaced (horizontally and/or vertically) cameras.

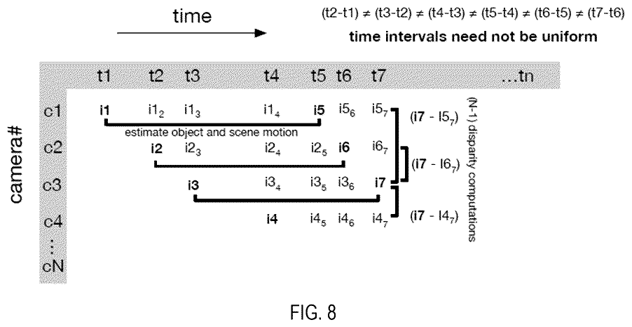

The following algorithm will be used to process the images taken from camera1, camera2, camera3, camera 4, . . . cameraN (see FIG. 8), where:
camera1 (c1) takes an image at time1 (t1)
c2 takes an image at t2 (t2>t1)
c3 takes an image at t3 (t3>t2)
cN takes an image at tN (t1<t2< . . . . <tN)
the staggering of image acquisition is such that time intervals between images (t1, t2, etc.) need not be equal. It is therefore possible that: (t2−t1)≠(t3−t2)≠ . . . ≠(tN−t[N−1]).

Non-uniform time intervals yield a natural anti-aliasing effect on parameter estimates derived from the time-series:
a. An image is obtained from c1 at t1 (image i1), c2 at t2 (image i2), and so on
   i. The characteristics of the current image, compared to previous images (particularly the previous image from the same camera, such as i1 and i5 in the diagram) are used to estimate the state of objects in the scene (state=position, velocity, luminance, etc.)
b. The state estimate is used to predict the image that would be acquired by any camera at later time points
   i. for example, the image i2 time-advanced from t2 to t3,t4, and so on (images i2₃, i2₄)
c. At each timepoint, the current camera image (e.g., i7 at t7) is compared to the time-advanced image predictions from all other cameras (e.g., i4₇, i5₇, i6₇; see FIG. 8).
   i. each of these (N−1) comparisons yields a disparity (stereo) depth map
d. Hyper-disparity (depth computed from multiple pairs of cameras with different geometrical configurations) is computed from the (N−1) disparity maps
   i. the weighting of the depth-map computed from i7 & i4₇ may not necessarily be equal to the weighting given to individual depth-maps computed from i7 and from i6₇ in the overall hyper-disparity depth mapping
   ii. different parts of the hyper-disparity depth mapping of pixels in i7 may be computed from 1, 2, . . . , or N−1 individual disparity mappings, depending on the overall geometrical configuration of the N cameras and the extent to which images from each will overlap

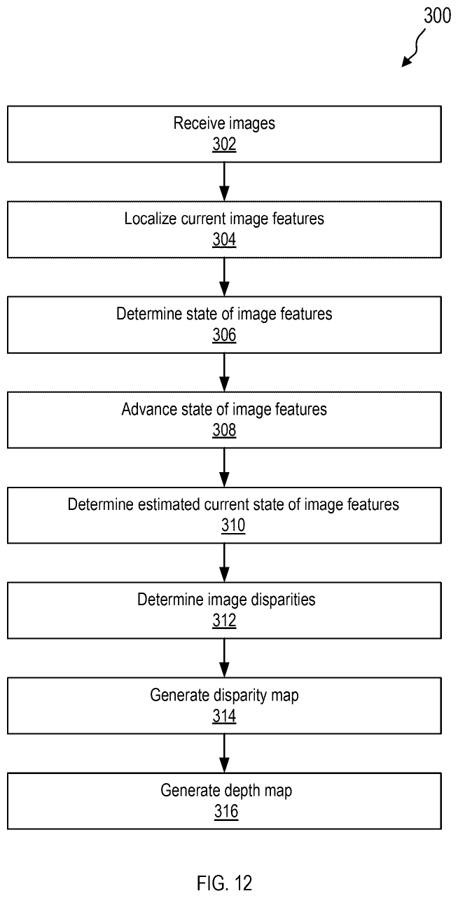

Referring to FIG. 12, a method 300 is shown. At 302, camera images are received. The images include two or more image sets, each image set acquired from a different camera. At 304, estimates of camera projections in real space are determined, and image features are localized. At 306, a state of image features is determined by comparing characteristics of current images to previously acquired images. At 308, the state of image features for the previously acquired images is advanced based on state parameters corresponding to the previously acquire and determining an estimated current state of the image features for the previously acquired images. At 310, estimated current positions of the image features based on the estimated current state of image feature are determined for the previously acquired images. At 312, image disparities for each image feature are determined comparing current positions of the image features in an image set of the two or more images sets to estimated current positions of the image features in a different image set. Then, corresponding image features within images of different image sets are identified, and spatial offsets of current positions of the image features and estimated current positions of the image features of the corresponding image features in each image set are determined. At 314, a disparity map is generated from the image disparities. At 316, a depth map is generated from the disparity map.

Eyetracking with 3D Fusion for Improved Tracking Ability, Stability, and Precision.

Calibration takes image features from eyetracking cameras and determines a gaze location of the eye, projected out into real space. Current image features used are 2D (e.g., image position of the pupil, image position of the limbus, image position of the corneal reflections). The current state of the art in eyetracking makes use of 2D features of the eye's image (such as, acquired by an eyetracking camera) to estimate the locus of gaze (e.g., eye fixations) in the world. Embodiments described herein include estimation processes using image features localized within images acquired by the eyetracking camera, and applies a calibration-mediated mapping between the state of the image features and real-world gaze positions. Techniques of the embodiments described herein provide a 3D image feature, the shape/orientation/position (i.e., “state”) of the 3D corneal surface, for use in estimating real-world gaze positions and/or directions (i.e., “eyetracking”), as well as a fusion of image-feature estimates, including corneal surface state and one or more 2D image features, for improved eyetracking.

Another embodiment described herein relates to systems and methods utilizing a sensor fusion technique combining a 3D depth map of a corneal surface with estimates that rely on pupil position, corneal reflection, and limbus tracking. Tracking the position, orientation and shape of the corneal surface provides an independent estimate of gaze for a fusion of estimates and an input to models of the eye, such as those used in automatic calibration procedures, such as the automatic calibration stage 110.

Lid closure (e.g., blinking) is a major source of data loss during eyetracking, particularly in patient populations exhibiting high blink rates (e.g., children, populations exposed to certain environmental factors, and patients with neurological conditions, psychiatric conditions, dry eye syndrome, medication side effects, etc.). Blink removal from data traces is an important feature of extant eyetracking algorithms. The ability to utilize, rather than remove (e.g., discard, filter, delete, etc.), data including blinks, can improved information gleaned from eyetracking.

In addition, partially-closed eyelids can render conventional eyetracking systems based only on limbus, pupil and/or corneal reflection inoperable, due to the lid substantially covering the eye and/or reflection feature tracked by these conventional eyetracking systems. The 3D corneal surface shape provides a new input to models of the eye that is not affected by eyelid closure and partial eyelid closure. Lid partial and full closure can make conventional eyetracking features (pupil limbus, corneal reflection) unusable. Advantageously, the technique described herein provides an input to eyetracking that can be obtained whether the eye is open, closed, or partially closed.

Typically, eyetracking is performed based on camera-image estimates of the positions of light-source reflections off of the eye and/or estimated pupil position. If torsion is tracked, iris markers or blood vessels are also monitored.

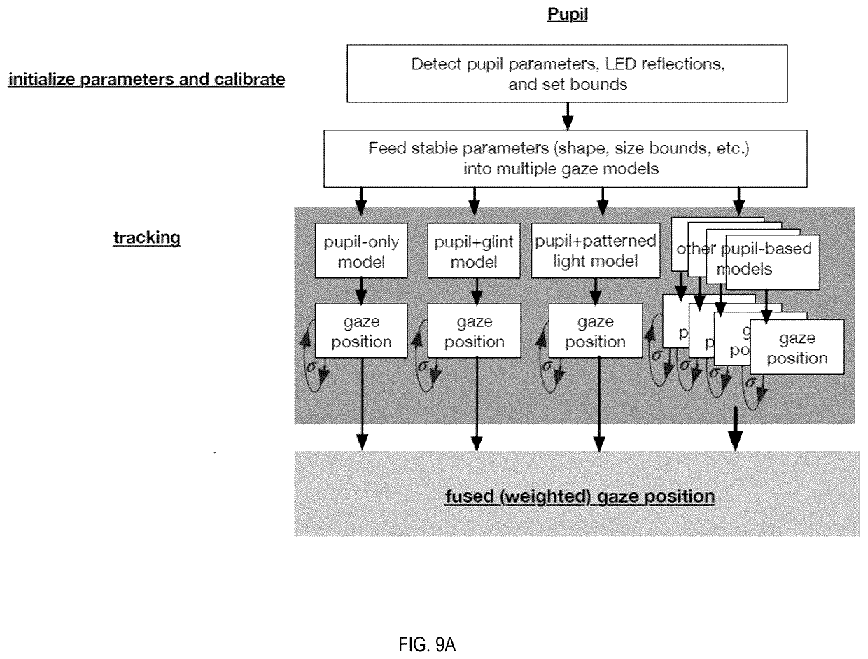

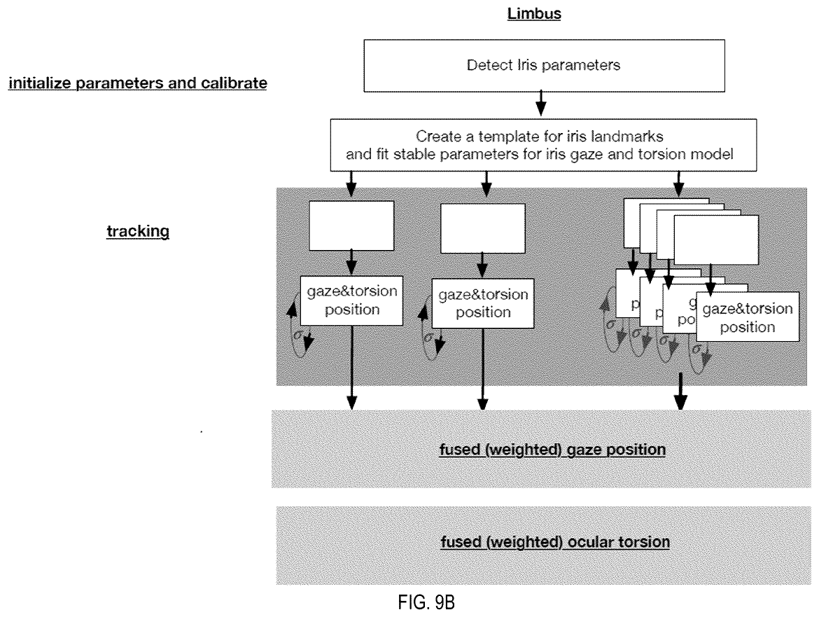

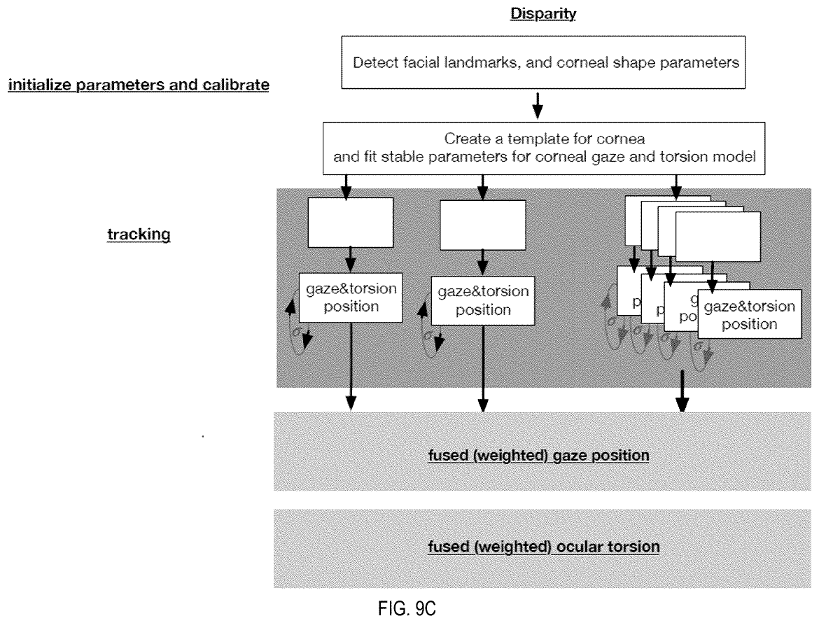

In one embodiment, a sensor fusion approach is used to estimate gaze (see FIG. 9). The fused estimates will be based on the information provided by three general classes of tracked entity:
1. Pupil (“dark pupil” and/or “light pupil”) either alone or in combination with positions of point reflections from infrared illumination (“corneal reflections”)
   i. there are multiple extant algorithms based on:
      1. light pupil (on-axis led light source reflects back from retina)
      2. dark pupil (off-axis led light source reflects iris but not pupil)
   ii. model-based estimates of gaze with pupil position as an input to the model
2. Limbus (visible-spectrum and/or IR spectrum illumination)
   i. track pupil position
   ii. track torsion
3. 3D corneal shape (multi-camera)
   i. track pupil position
   ii. track torsion

Those skilled in the art will appreciate that there are many well-known algorithms that can be used under (1) and (2). For (3), the 3-D shape of the cornea is computed from disparity, and the raw depth map can be template matched to a corneal model in various poses (gaze directions). There are many template matching algorithms known in the art, such as Bayesian parameter estimation, in which a model (idealized corneal shape) with parameters for the specifics of corneal curvature, gaze direction (azimuth, elevation) and torsional state are fitted, yielding an estimate of the gaze direction, corneal shape, and torsional state. In the Bayesian algorithm, the prior state (fitted values) can be used to improve the current estimate, and nuisance variables (e.g., the exact corneal shape) can be marginalized out of the final gaze estimate.

Corneal curvature is also used in models of the physical eye and its optics (“gaze model”), which allow for a fused estimate of these models. E.g., if corneal curvature acts as a parameter in a gaze model, then having a traditional 2D-based estimate fused with the 3D disparity-based estimate will provide a better overall fit and stability-of-fit to the gaze model.

Stability is improved with a sensor fusion approach because each estimate is based on a different type of information. When one is unreliable (or unavailable), the others take over the fusion through a dynamic weighting process (e.g., based on inverse signal variance).

Precision is improved because when more than one information source (regarding gaze) is available, the combined estimate of gaze direction is more precise than from any single source in isolation.

The tracking ability of this apparatus is improved relative to current eyetrackers, as determining and monitoring 3D corneal shape gives a new input to the gaze estimation fusion algorithm, and allows the tracker to estimate gaze direction when the eye is closed, given that the lid adheres to the cornea and causes the skin of the lid to take on approximately the same 3D shape and will provide the same information regarding gaze as the cornea itself in the open eye.

Outputs of the tracker include: time courses of gaze direction, eyelid position, pupil diameter, and ocular torsion, despite lid closure (i.e., gaze, pupil, torsion etc. output estimates are available whether the eyelid is open, closed, or partially closed).

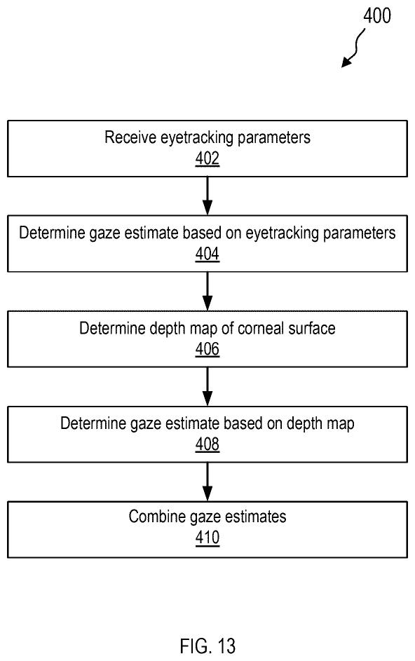

Referring to FIG. 13, a method 400 is shown. At 402, eyetracking parameters associated with pupil, limbus, and corneal reflection of an eye are received from two or more cameras. At 404, a gaze estimate based on the eyetracking parameters associated with pupil, limbus, and corneal reflection is determined. At 406, a depth map of a corneal surface of the eye is determined. At 408, a gaze estimate based on the depth map is determined. At 410, the gaze estimate based on the eyetracking parameters associated with pupil, limbus, and corneal reflection and the gaze estimate based on the depth map are combined, forming a fused gaze estimate. In some embodiments, gaze estimation based on corneal state, such as at 406 and 408, may be used alone, and not combined with other image-features and their corresponding gaze estimates.

### EXPERIMENTAL EXAMPLES

The following section describes experimental methods relating to new eyetracking calibration techniques. These examples are merely illustrations and should not be construed as limiting the disclosure.

### Free-Viewing Calibration Study

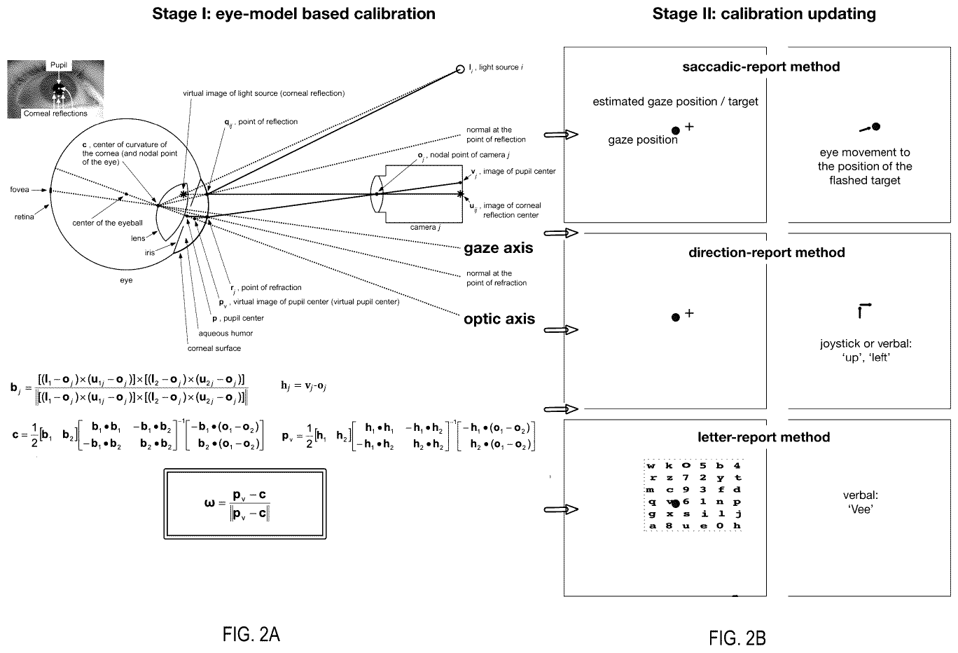

Free-viewing calibration is designed to be useful even in cases when subjects cannot maintain prolonged fixation, have reduced ocular range of motion, or involuntary eye movements. FIGS. 2A-2B describe the new algorithm. First, an initial coarse calibration is computed from a model of the eye (FIG. 2A), allowing us to place a target on the screen during fixation at the gaze estimate (FIG. 2B, left). A target flashed at the gaze estimate then allows the observer to provide an error signal (FIG. 2B, right) that the algorithm uses to correct the calibration (and future gaze estimates).

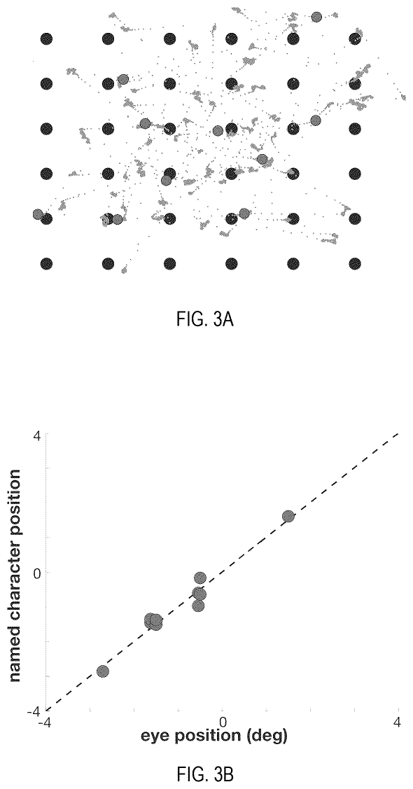

FIG. 3A shows eye movements while free-viewing a portion of the screen. During periods of fixation, a punctate target, or grid of characters (FIG. 2B, lower left) is flashed. Raw eyetracker output is precisely known during these brief flashes. Subjects either report the letter closest to the point of regard, or saccade to the flashed target.

Free-viewing calibration methods rely on the fact that one can respond to briefly flashed stimuli near fixation. This is obviously true of the saccadic-report method (FIG. 2B, top); without actively inhibiting such movement, observers reflexively turn the eye to look at a flashed target. The direction-report method effectively performs a binary search in x- and y-dimensions (a highly efficient search algorithm) and is expected to easily outperform standard calibration techniques. Finally, the letter-report method is similar in important respects to Sperling's “partial report method”. The partial report method uses a 50 ms flashed grid of letters to probe memory. Sperling's method has been used successfully in many populations including with the elderly, and adults with mild cognitive and attentional impairment. Unlike our method, Sperling's method requires the additional difficulties of reporting multiple letters and employs a post-grid visual mask. Our procedure will be substantially easier to perform than Sperling's partial report, greatly reducing the risk of any difficulties acquiring calibration data in control and patient populations.

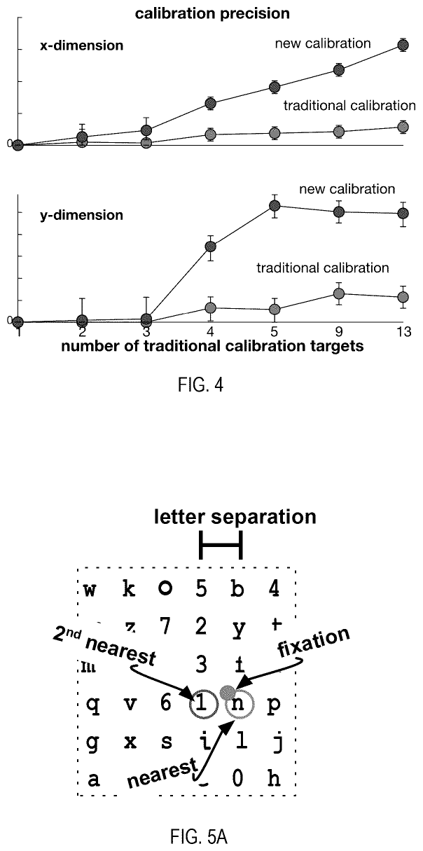

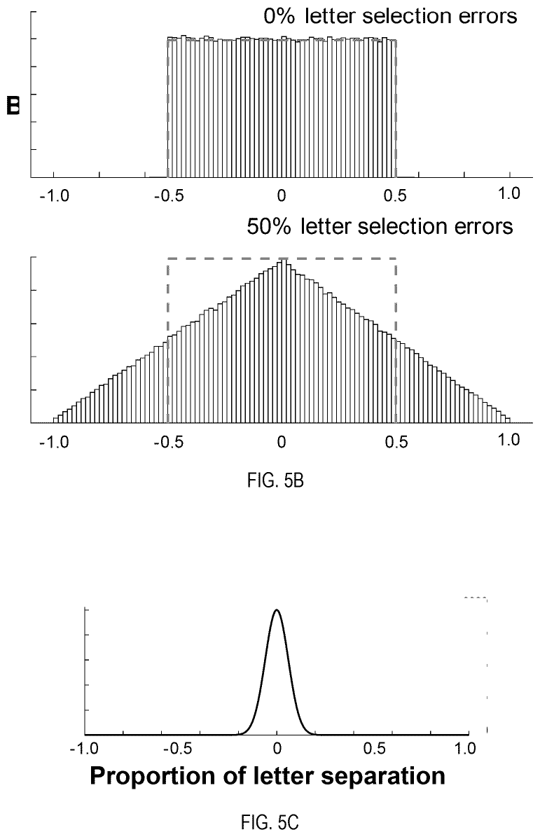

Pilot data (FIGS. 3A-3B) with the letter-report method shows promising results, even with a modest number (10) of letter-report/fixation pairs. Further, simulations show that mappings are highly accurate (FIGS. 4, 5A-5D). Even with 50% errors in reporting the nearest character to gaze center (FIGS. 5B-5C), the sampling distribution of errors is triangular (first convolution of the uniform, “no identification errors”, distribution), and becomes a narrow Gaussian (central limit theorem) when multiple reports are combined (FIG. 5C). It is further expected that this participant-driven procedure (self-paced based on their own fixations) will enhance engagement and improve attention to the task. Improved attention will, in turn, further improve data quality relative to standard methods.

This combination of pilot and simulation results demonstrates feasibility with regard to: (1) data acquisition, (2) precision per unit time spent collecting data, and (3) robustness to errors.

### A. Research Design

To execute algorithm/method development, experiments examined the effects of different implementations of the algorithm—both the post-flash report method (FIGS. 2A-2B) and the update algorithm—on its precision (minimizing overall mapping error) and efficiency (time to threshold precision). These variations will be tested against the standard calibration (EyeLink 13-point built-in). Twenty-seven control (25 in Aims 1, 2 adjusted for dropout) and 48 patients with Parkinson's n disease and atypical parkinsonism (40 in Aims 1, 2 adjusted for dropout) will be enrolled. See Human Subjects for in-/exclusion.

### B. Study Procedures

Subjects will be instructed on the study procedure, screened to ensure they meet study criteria, complete informed consent, and seated comfortably in front of a computer monitor in an adjustable-height chair. Subjects will be asked to look at the screen, which may contain a static or dynamic image to increase visual interest and encourage visual exploration during free viewing.

In Phase I a coarse calibration is obtained, following FIG. 2A. Here an adaptive threshold is computed for saccade detection, which will be used in the next phase to identify saccades and fixations in real time. Coarse calibrations are refined in Phase II. A circular target, crosshairs, or letter-grid is flashed periodically during fixation at the estimated fixation position (FIG. 2B). Subjects make the required post-flash report (saccadic or verbal) at their leisure. This sequence is repeated for 5 minutes (or a minimum of 30 post-flash reports).

Compare three post-target response methods to conventional calibration. The new calibration will proceed in two phases: the first phase is designed to obtain a coarse calibration mapping (FIG. 2A). This serves as the Bayesian prior in the second phase (FIG. B). The prior is computed using the geometry relating corneal reflections, entrance pupil position, and the optic axis (FIG. 2A). This type of prior contains on the order of ˜10° error, and must be corrected to obtain a useful calibration. Correction can be effected via any one of the free-viewing methods outlined in FIGS. 2A-2B, but each has different strengths and weaknesses, and it is important to understand how these interact with ocular motor pathology for designing a final implementation that can accommodate different disorders.

The saccadic-report method has the potential to be the fastest and easiest-to-use method, because the instruction is simply to look at any onscreen flashed targets as they appear, and this occurs reflexively. The direction-report method can be implemented as a saccadic, joystick or verbal report. Reporting just the direction will be useful for PSP patients, whose saccades are highly dysmetric. The letter-report method (described above as a variant of Sperling's partial-report method) offers a compromise between the universal applicability of the direction-report method and the advantage of metric data generated by the saccadic-report method. In some embodiments, saccadic report may be accomplished with a continuously-visible target rather than a flashed target.

Letter-report data was collected (pilot data; FIGS. 3A-3B) in a healthy control with a stationary, central grid. These data show it is possible to obtain a reasonable first approximation to the correct mapping, even with a modest number of letter-report/fixation pairs (10 report/fixation pairs). Simulations further show that mappings are highly accurate (FIGS. 4, 5A-5D)—even when errors are made during letter-naming. FIGS. 5B-5C demonstrate, with 50% errors in identifying the nearest character to gaze center, the sampling distribution of errors is triangular (first convolution of the uniform distribution describing the errorless case). This becomes a narrow Gaussian (central limit theorem) when combining data from multiple fixations (FIG. 5C).

Compare two types of updates. Gaze is initially predicted based on the geometry of the eye and entrance pupil relative to the eye's optic axis. Although the optic axis of the eye is several degrees rotated relative to the visual or gaze axis of the eye (FIG. 2A), both in the horizontal and vertical dimensions, it can serve as an initial coarse estimate of gaze direction. The error in model-based estimates of gaze direction (equating gaze and optic axes) comes from several sources: As mentioned, there are interindividual differences that make it impossible to correct model-based estimates of gaze direction without additional subject-specific calibration data. In addition, these errors change with the orientation of the eye and the state of accommodation, which both change the relation of entrance pupil to the aperture stop created by the iris (FIG. 2A). These variables are also affected by the curvature of the cornea, which varies substantially among individuals, the rotational symmetry of cornea and lens anterior surfaces, and the refractive power of an individual's lens-cornea-vitreous/aqueous-humor optical system.

In one embodiment of updating that may be used in Stage II, “Model-update” computations use data from one of the error-report methods (FIG. 2B) to correct the model of the eye itself. Using wide priors on the angles separating optic and gaze axes, center of corneal curvature, center of rotation, foveal position etc. (depending on the complexity of the eye model). Bayesian updating is used to estimate the separation of gaze vs optic axes of the eye at the anterior nodal point, marginalizing over all other model parameters. The model-update method is computationally complex, but yields onscreen gaze estimates for any incoming camera datum—without the need for new estimates to be interpolated from a discrete set of screen calibration points. In another embodiment of updating that may be used in Stage II, “Fitted-update” computations use a discrete set of screen calibration points to construct a mapping between the pupil-to-glint vector and gaze direction. In this method a smooth, interpolatable function is fit to the pupil-glint vs screen position data. In another embodiment of the updating that may be used in Stage II, a fusion of “Fitted-update” and “Model-update” computations may be employed.

### C. Data Analysis and Expected Outcomes

Saccadic report method. The Bayesian updating used to improve coarse Phase I calibrations (FIG. 2A) uses a likelihood describing saccade direction and amplitude, given target direction and distance (i.e., p(θ·α|θ′·α′·t)). This 2D likelihood must encode saccadic hypometria for larger saccades.

Direction-report method. A likelihood similar was employed to that used for measuring a sensory threshold (Hudson), which will be identical whether using a saccade, verbal or joystick-based report.

Letter-report method. Likelihoods for letter reporting are shown in FIGS. 5A-5D.

All versions of the model-update algorithm require iteratively updating the eye-model based on post-flash reports. For example, in the saccadic-report version of model update, the post-flash saccade is used to provide data regarding the error in screen position predicted by pupil position, given the current set of model parameters—allowing those parameters to be iteratively updated following each fixation/report cycle.

By comparison, in all versions of the fitted-update algorithm, the basic procedure will involve computing a mapping between raw tracker data to screen locations. These data require an “errors in the variables” rather than a regression fitting procedure because regression demands errorless values on the abscissa. As an example, the solution to the errors in variables fitting problem for polynomials of degree 1 is derived by Hudson. This solution is the Bayesian posterior over possible values of the slope parameter (α=tan φ; which defines a line at an angle φ to the abscissa) and bias term (β). The Bayesian posterior is computed after integrating over the unknown true screen-eye position pairs (i.e., true x-y locations) and the unknown standard deviation (σ) of the observations:

$$p ( φ \cdot β \vert d ) ∝ ϖ ( φ ) {\int }_{0}^{∞} d σ ϖ ( σ ) {\int }_{- ∞}^{∞} d {ℓ}_{1} … d {ℓ}_{N} p ( ℓ \vert φ ) p ( d \vert φ \cdot ℓ \cdot σ ) ∝ {\int }_{0}^{∞} \frac{d σ}{σ} {\int }_{- ∞}^{∞} d ℓ {σ}^{- 2 N} {e}^{- \frac{1}{2 {σ}^{2}} {\sum }_{k} [ {( {d}_{1 k} )}^{2} + {( {d}_{2 k} - β )}^{2} + {ℓ}^{2} - 2 {ℓ}_{k} ( {d}_{1 k} cos φ + [ {d}_{2 k} - β ] sin φ ) ]} ∝ ϖ ( φ \cdot β ) {\overline{{ϵ}_{ℓ}^{2}}}^{- \frac{N}{2}}$$

where [symbol] is the set of “true” screen-gaze positions on the calibration line, ε [symbol] is the corresponding set of errors in the observed noisy screen-gaze position data [i.e., ϵ [symbol] ₖd₁ₖ sin φ−(d₂ₖ−β)cos φ]. Jeffreys' priors are assigned to both unknown parameters, ω̅(φ)∝dφ and ω̅(σ)∝dσ/σ. ω̅(β) is set to a bounded uniform to match the known range of possible screen positions and raw tracker outputs. Note that a uniform prior in slope [α] is not used because it has the undesirable property that it biases the result toward zero. Jeffreys' priors are the most conservative choices of priors.

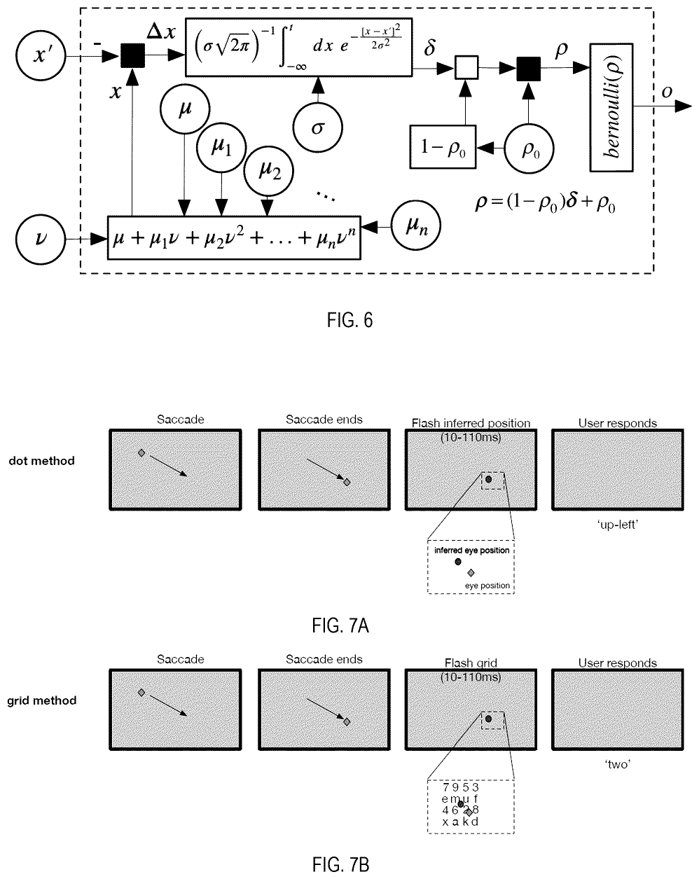

When paired with the direction-report update the process required a more complex analysis, in that binary search/thresholding first finds the 50% point of direction error. This first step is overlaid on the polynomial mapping fit (in both x, y). The graphical model describing the underlying generative binomial probability and polynomial fit is shown in FIG. 6, where x′ is true gaze location, v is the pupil-glint vector, and the output (o) is a binary left/right or up down.

Also, d-prime is measured for correct letter identification as a function of presentation time, and in particular interrogate any differences between control and patient populations. Shorter presentation times will be preferred in the final algorithm because the eye position given by raw eyetracker data is less precisely known as presentation time increases.

### D. Alternative Embodiments

It is anticipated that free-viewing a blank screen may seem odd/boring, and the system can utilize one or more screensaver-style backgrounds (e.g., static geometric images, twinkling lights, moving fractals, etc.) to enhance attention/decrease boredom while visually exploring the screen.

The direction-report method offers saccade, verbal and joystick options of report modality. Although the first option is clearly the most efficient, saccadic dysmetria may limit this post-flash reporting mode's usefulness for certain patients. It is also possible that patients displaying a large number of saccadic intrusions, or patients with nystagmus, will not be able to generate saccadic responses that can be distinguished from involuntary eye movements. In these cases, a verbal or joystick-based response mode will be preferred.

If significant outliers are observed in the character/eye position data, outlier suppression can be emulated, modeling data as generated by two signals: a low-variance signal representing the bulk of the data, and a high-variance signal that yields outliers:

$$ϖ ( σ ) = p ( σ \vert {σ}^{′} \cdot θ \cdot γ ) = θ [ δ ( σ - γ {σ}^{′} ) ] + ( 1 - θ ) [ δ ( σ - {σ}^{′} ) ]$$

This prior essentially models eyetracker output as a biased coin-flip (binomial rate) between occasional outliers and normal-variance data. The solution automatically down-weights the contribution of high-variance data and effectively ignores outliers.

### Definitions

No claim element herein is to be construed under the provisions of 35 U.S.C. § 112(f), unless the element is expressly recited using the phrase “means for.”

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any references herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

As used herein, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A system, comprising:
one or more processors; and
one or more memory devices operatively coupled to the one or more processors, the one or more memory devices having instructions stored thereon which, when executed by the one or more processors, cause the one or more processors to perform operations comprising:
detecting, in camera images, ocular landmarks of a subject's eye;
determining ocular image parameters based on the ocular landmarks;
determining a coarse calibration output associated with the ocular image parameters;
detecting a first eye movement made by the subject, the first eye movement associated with a first gaze direction;
determining a first landing position of the eye at an end of the first eye movement, the ocular landmarks at the first landing position associated with a first gaze location on a screen;
displaying, on the screen, a target at the first gaze location;
receiving, responsive to the target being displayed at the first gaze location, a first input from the subject, the first input including a response relating the first landing position to the first gaze direction; and
updating the coarse calibration output based on the first input.

2. The system of claim 1, wherein the instructions further cause the one or more processors to:
detect a second eye movement made by the subject, the second eye movement associated with a second gaze direction;
determine a second landing position of the eye at the end of the second eye movement, the second landing position associated with a second gaze location on the screen;
display, on the screen, the target at the second gaze location;
receive, responsive to the target being displayed at the second gaze location, a second input from the subject, the second input including a response relating the second landing position to the second gaze direction; and
update the coarse calibration output based on the second input.

3. The system of claim 1, wherein the ocular image parameters are associated with a location and direction of an optic axis.

4. The system of claim 3, wherein determining the coarse calibration output comprises:
generating a set of landmark positions;
determining a set of screen locations associated with the set of landmark positions; and
determining a mapping between the set of landmark positions and the set of screen locations.

5. The system of claim 1, wherein the ocular image parameters are associated with determining a fit of a gaze model.

6. The system of claim 5, wherein determining the coarse calibration output comprises fitting the ocular image parameters of the gaze model.

7. The system of claim 1, wherein displaying the target comprises flashing a dot at the first gaze location, and the response comprises an indication of a direction of the dot relative to an actual gaze position.

8. The system of claim 1, wherein displaying the target comprises flashing a grid of unique characters centered at the first gaze location, and the response comprises an indication of a character in the grid of unique characters proximate to an actual center of gaze.

9. The system of claim 1, wherein updating the coarse calibration output is based on Bayesian updating.

10. The system of claim 1, wherein the instructions further cause the one or more processors to determine a gaze estimate based on an optic axis of the subject.

11. The system of claim 10, wherein determining the first gaze estimate is based on a model of the eye.

12. The system of claim 11, wherein the instructions further cause the one or more processors to update the gaze estimate by estimating a separation angle between the optic axis and a gaze axis of the subject.

13. The system of claim 11, wherein the instructions further cause the one or more processors to update the gaze estimate by constructing a mapping between pupil-glint positions and a set of screen calibration points.

14. A system, comprising:
one or more processors;
two or more cameras; and
one or more memories operatively coupled to the one or more processors, the one or more memories having instructions stored thereon which, when executed by the one or more processors, cause the one or more processors to perform operations comprising:
receiving images, the images comprising two or more image sets, each image set acquired from a different camera
determining estimates of camera projections in real space and localizing image features;
determining a state of image features, by comparing characteristics of current images to previously acquired images;
advancing the state of the image features for the previously acquired images based on state parameters corresponding to the previously acquired images, and determining an estimated current state of the image features for the previously acquired images t;
determining estimated current positions of the image features based on the estimated current state of the image features for the previously acquired images;
determining image disparities for each image feature by comparing current positions of the image features in an image set of the two or more image sets to estimated current positions of the same image features in a different image set of the two or more image sets;
identifying corresponding image features within images of different image sets;
determining spatial offsets of current positions of the image features and estimated current positions of the image features of the corresponding image features in each image set;
generating a disparity map from the image disparities; and
generating a depth map from the disparity map.

15. The system of claim 14, wherein generating the disparity map comprises combining a single current image with a single previously acquired image based on the previously acquired images and the estimated current positions of the image features for the previously acquired images.

16. The system of claim 14, wherein generating the disparity map comprises combining a single present image and estimates based on the previous acquired images and the estimated current positions of the image features.

17. A system, comprising:
one or more processors; and
one or more memories operatively coupled to the one or more processors, the one or more memories having instructions stored thereon which, when executed by the one or more processors, cause the one or more processors to perform operations comprising:
receiving, from two or more cameras, eyetracking parameters associated with pupil, limbus, and corneal reflection of an eye;
determining a gaze estimate based on the eyetracking parameters associated with pupil, limbus, and corneal reflection;
determining a depth map of a corneal surface of the eye;
determining a gaze estimate based on the depth map; and
combining the gaze estimate based on the eyetracking parameters associated with pupil, limbus, and corneal reflection and the gaze estimate based on the depth map, forming a fused gaze estimate.

18. The system of claim 17, wherein the eyetracking parameters include track-pupil position and track torsion.

19. The system of claim 17, wherein the instructions further cause the one or more processors to determine at least one of gaze direction, eyelid position, pupil diameters, and ocular torsion.

20. The system of claim 17, wherein the fused gaze estimate is based on a dynamic weighting process.
